(19) United States
(12) Patent Application Publication
(10) Pub. No.: US 2025/0087962 A1
(43) Pub. Date: Mar. 13, 2025

(54) SLOW LIGHT AMPLIFIER AND METHODOLOGY FOR IMPROVING SIGNAL STRENGTH IN ACOUSTO-OPTICAL TOMOGRAPHY

(71) Applicant: DEEP LIGHT VISION AB, Lund (SE)

(72) Inventors: Stefan KRÖLL, Dalby (SE); Lars RIPPE, Lund (SE)

(73) Assignee: DEEP LIGHT VISION AB, Lund (SE)

(21) Appl. No.: 18/715,104

(22) PCT Filed: Dec. 1, 2022
(86) PCT No.: PCT/EP2022/084147

§ 371 Date: May 30, 2024

(30) Foreign Application Priority Data
Dec. 1, 2021 (SE) 2151467-4

(51) Int. Cl. H01S 3/23; H01S 3/10

(52) U.S. Cl. CPC H01S 3/2316; H01S 3/10023; H01S 3/235; H01S 2301/02

(57) ABSTRACT

A cascaded slow light amplifier and a system comprising such amplifier is described. The amplifier includes a unit wherein each unit comprising of at least one host crystal doped with ions and optically prepared to provide at least one amplification

### BACKGROUND OF THE INVENTION

### Field of the Invention

This disclosure pertains in general to a method for increasing the detected signal in the acousto-optical tomography scheme. In particular, the disclosure pertains to how the signal may be greatly amplified while limiting noise from amplified spontaneous emission. Furthermore, the disclosure also pertains a methodology for recoupling the signal light back into the imaging medium allowing for an even greater signal amplification via mode selection.

### Background of the Disclosure

The ability to shine light into turbid, i.e., light scattering, media is important in many settings, including but not limited to biomedical applications. Optical imaging provides highly sensitive molecular contrast in a simple and non-invasive manner. Traditional light microscopy relies on penetration of light through shallow layers, in the order of tens of micrometres. Confocal microscopy techniques extend the penetration depths up to in the order of 0.1 mm, while optical coherence tomography can reach to depth in the order of 0.5 mm. This is approximately the limit of high-resolution optical imaging modalities, which rely on the suppression of diffusely scattered light. At larger depths, diffuse light scattering dominates the interaction of the light field and the medium. The attenuation of an impending light beam at a certain depth is largely determined by the scattering of the medium. This fundamental aspect means that even at the most favourable wavelengths, the maximum penetration depth where light is detectable is limited to in the order of 10 cm in typical biological tissue.

In the diffuse scattering regime, from about a millimetre up to several centimetres, other optical analysis and optical imaging modalities have been developed. One example is pulse oximetry for determination of blood oxygen saturation. One example of optical imaging is diffuse optical tomography, which can be performed, for example, with endogenous tissue contrast, with added contrast agents, in fluorescence, or with added fluorescing agents. Generally, because of the diffuse scattering, spatial localization is limited with these methods to in the order of several millimetres up to a centimetre or more. In addition, because of the very low light intensities in the detected light, the detection schemes are often cumbersome, slow, and require advanced and expensive equipment.

To improve the spatial localization when probing turbid media with light, photoacoustic schemes have been devised. In photoacoustic tomography, acoustic waves that emanate locally, due to slight heating in the tissue due to absorption of laser pulses, are detected by ultrasound transducers. Photoacoustic tomography takes advantage of the fact that ultrasound waves are scattered several orders of magnitude less than light waves in tissue. Thus, the spatial localisation of the origin of the acoustic waves can be in the order of a millimetre or less.

Another example of the use of acoustics to improve localisation of light interaction in tissue is the use of acoustic photon tagging, as described for example in J. Gunther & S. Anderson-Engels (2017 October), Review of current methods of acousto-optical tomography for biomedical applications, Frontiers of Optoelectronics, 10 (3), 211-238. By insonifying a volume in the tissue, and directing laser light into the tissue, only the laser light that passes through the region occupied by an acoustic field is frequency-shifted by the acoustic frequency. By optical detection of the frequency-shifted light only, it can be known that that light has interacted with the acoustic field, which compared to the light can be made to occupy small and localized volume.

However, this acousto-optical tomography scheme is still limited by the amount of light which reaches and interacts with the acoustic field. Hence, methods and apparatuses for improving the amount of light interacting with the ultrasound focus would be advantageous.

This increased light intensity can be achieved via wavefront shaping, which allows for several orders of magnitude higher intensities at the desired locations, as described in I. M. Vellekoop and A. P. Mosk, “Focusing coherent light through opaque strongly scattering media,” Opt. Lett. 32, 2309-2311 (2007). An analog way of performing this wavefront shaping is via mode competition in roundtrips through in an optical amplifier, as described in M. Nixon, O. Katz, E. Small, Y. Bromberg, A. A. Friesem Y. Silberberg and N. Davidson, “Real-time wavefront shaping through scattering media by all-optical feedback”. Nature Photon 7, 919-924 (2013).

Another type of optical amplifiers are cascade amplifiers, or multistage amplifiers, wherein each stage uses a doped crystal to amplify the signal. These types of amplifiers are described in, for example CN110895377 A; U.S. Pat. No. 5,872,650 A; JP2010205903 A; and US2003156605 Al.

An iterative scheme which amplifies only the shifted light would thus quickly increase the optical field strength at the point of acousto-optical interaction. An issue which arises from amplification of the scattered light which is collected from the tissue is that amplified spontaneous emission (ASE) cannot be rejected without also rejecting a sizeable amount of signal. Traditional countermeasures for ASE include using low amplification or inclusion of an angular selection stage, both of which are detrimental in the described application.

### SUMMARY OF THE INVENTION

Accordingly, examples of the present disclosure preferably seek to mitigate, alleviate or eliminate one or more deficiencies, disadvantages or issues in the art, such as the above-identified, singly or in any combination by providing a device, system or method according to the appended patent claims for slow-light amplification and improving signal strength in acousto-optical tomography.

A first aspect of the disclosure relates to cascaded slow light amplifier. The amplifier includes a unit wherein each unit comprises of at least one host crystal doped with ions and optically prepared to provide at least one amplification zone.

In some examples may the unit include at least one time-filtration zone.

In some examples may each zone be made of a single crystal and wherein the unit is obtained by joining at least two crystals.

In some examples may a single crystal include the at least one amplification zone and the at least one time-filtration zone.

In some example may the amplifier comprise of a chain of a plurality of units being linked.

In some example may the chain include a plurality of the amplification zones and a plurality of the time filtration zones, wherein each of the amplification zone is subsequently followed by one of the time-filtration zones. In some examples may the chain be made from a single crystal.

In some example may a final time filter in the chain acts as a frequency shifter.

In some examples may the amplification zone be a narrowband amplifier.

In some examples may the amplification zone be a two-level amplifier.

In some examples may the amplification zones in the chain be sequentially pumped and the amplification zones in the chain has an amplification level where a spontaneous emission (ASE) is lower than an incoming signal.

In some examples may the time-filtration zone, which may be arranged between two amplification zones, act as a time buffer.

In some examples may the amplification have a strong suppression outside a gain bandwidth and with enhanced slow light effect.

In a further aspect of the disclosure is a system for regenerating an amplifier stage described. The system includes a cascaded slow light amplifier according to the disclosure. The system may also include an electric source and connectors connected to the electric source and to the amplification zones of the cascaded slow light amplifier. The connectors may be arranged to provide an electric field over one or several the amplification zones for regenerating the amplifier stage using the electric field.

In another aspect of the disclosure, a system for localising light in light-scattering media is described. The system may include a light source configured for transmitting a light signal having a frequency into a light-scattering medium. The system may also include an ultrasonic device configured for generating an acoustic field in the light-scattering medium, and an optical subsystem comprising a system for regenerating an amplifier stage as herein described.

Further, the system may include collection light guides configured for collecting the light signal traversed through the light scattering medium. When in use, the system provides an amplifier roundtrip where the light signal traverses the light-scattering medium via different optical modes, wherein some modes interact with the acoustic field whereby the modes interacting with the acoustic field has a part of its power spectrum shifted in frequency, and the light collected by the collection light guides is transmitted to the optical subsystem wherein the system for regenerating an amplifier stage amplifies frequency shifted modes and suppresses all other modes before shifting the amplified modes back to the frequency of the light signal and thereafter transmitting it back into the light-scattering medium.

In some examples may a repetition rate of the ultrasonic device be timed with a time of the amplifier roundtrip.

In some examples may the repetition rate of the ultrasonic device be incrementally changed thereby shifting the acoustic field deeper into the light-scattering medium, whereby the amplified modes follow the ultrasound field deeper into the light-scattering medium for each of the amplifier roundtrip.

The term “slow light” refers to a propagating pulse being substantially slowed by the interaction with the medium in which the propagation takes place. “Slow light” may have a very low group velocity of light that is several orders of magnitude slower than the speed of light in vacuum, such as the group velocity may be smaller than thousands or millions of times less than the speed of light, c.

As an example, “slow light” may be have a group velocity smaller than c/10\hat{ }3, such as smaller than c/10\hat{ }4, where c (=3x10\hat{ }8 m/s) is the speed of light in vacuum. For example, this means that in a slow light amplifier, the group velocity of a light pulse undergoing amplification will be slower than 300 km/s, such as slower than 30 km/s.

It should be emphasized that the term “light” when used in this specification is not limited to light visible to the human eye, but also comprises wavelengths in the ultraviolet and infrared regions.

It should also be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which examples of the disclosure are capable of will be apparent and elucidated from the following description of examples of the present disclosure, reference being made to the accompanying drawings, in which

FIG. 1 is illustrating a schematic example of a setup for imaging using acoustic photon tagging with an optical amplification loop to boost signal levels;

FIG. 2 is illustrating a schematic structure of a cascaded slow light amplifier;

FIG. 3 is illustrating the absorption vs frequency for a slow light 2-level amplifier after population inversion; and

FIG. 4 is illustrating the absorption vs frequency of a slow light amplifier and how the amplification is continually regenerated by shifting ion populations using the Stark effect.

### DESCRIPTION OF EXAMPLES

Specific examples of the disclosure will now be described with reference to the accompanying drawings. This disclosure may, however, be embodied in many different forms and should not be construed as limited to the examples set forth herein; rather, these examples are provided so that this disclosure will be thorough and complete, and will fully convey the scope of the disclosure to those skilled in the art.

The following disclosure focuses on examples of the present optical subsystem 1-4 applicable to improve the localisation of light interaction in light-scattering media. The disclosure may be applicable for measurements of oxygen saturation deep inside living tissue, such as the brain, heart, female breast, or muscle tissue. However, it will be appreciated that the description is not limited to this application but may be applied to many other systems where localisation of light interaction in light-scattering media is useful.

The disclosure generally comprises a complement of the previously submitted Swedish patent application 2151298-3 called System and method for localising light in light-scattering media, herein incorporated by reference.

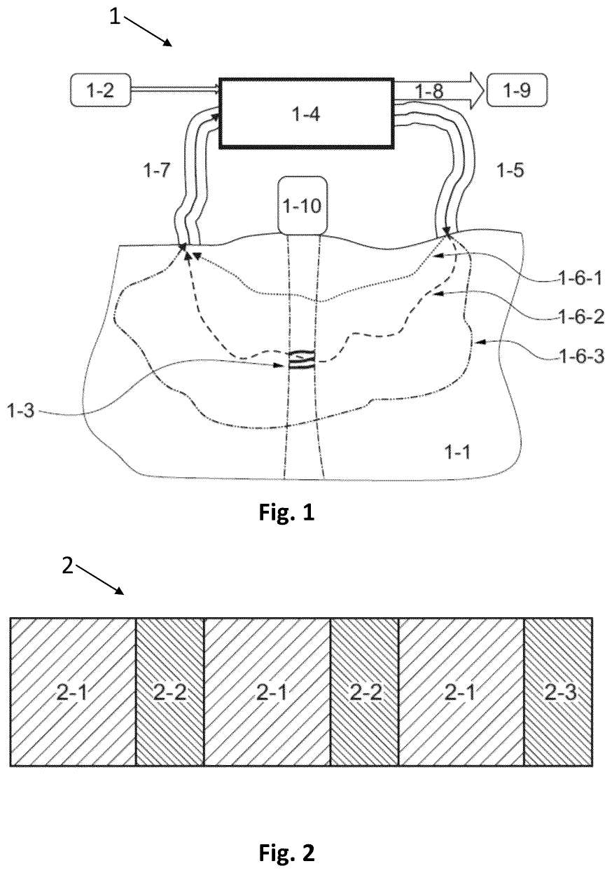

In the schematic illustration in FIG. 1, the system 1 is an example of the system specified in System and method for localising light in light-scattering media. Similar to the previous application, the system 1 includes an optical source 1-2 (e.g. laser) with frequency f1 and, an ultrasonic device 1-10 which emits an acoustic field 1-3 into the light-scattering medium 1-1 with frequency f2. The laser may be coupled into the tissue via an optical subsystem 1-4, which is the focus of this disclosure. The light out from the optical subsystem 1-4 may be directed into the light scattering-medium via the illumination waveguide 1-5 and into the light scattering medium. There the light traverses the medium via different optical modes 1-6, where some modes interact with the acoustic field 1-3, e.g. 1-6-2, and other modes do not, e.g. 1-6-1 and 1-6-3. The light which interacts with the acoustic field has a part of its power spectrum shifted in frequency to the frequency f3=f1+n*f2, where n is an integer. In turn, this light experience losses based on the local optical absorption at the acoustic field 1-3. The light may then be collected at the collection waveguide 1-7, guiding the light to the optical subsystem 1-4. The optical subsystem amplifies the frequency shifted light only before shifting it back to the original light source 1-2 frequency and re-injecting most of this light into the illumination waveguide 1-5. A small part of this light may however be ejected out of the optical subsystem 1-4 as a monitor signal 1-8 which may be monitored on the detector 1-9.

As the reinjected amplified light is intrinsically connected to optical modes 1-6 which traverses the acoustic field, multiple roundtrips of the light through the system loop 1-4 to 1-5 to 1-1 to 1-7 to 1-4 etc. favours optical modes 1-6 which traverses the acoustic field 1-3.

Furthermore, over multiple round trips, competition for the amplification in the optical subsystem 1-4 favours the modes 1-6-2 which has the lowest losses through the light scattering medium 1-1.

Both these effects, i.e. of only amplifying modes which interact with the acoustic field 1-3 and competition between these modes allows for more light to reach the acoustic field 1-3 for the same light intensity injected into the light scattering medium 1-1 through the illumination waveguide 1-5. This allows for a stronger signal readout. Furthermore, small movements of the acoustic field 1-3 in between roundtrips allows for this high intensity to follow the acoustic field inside the tissue. This allows for both a circumvention of waiting for the intensity to build up at different probe points inside the light-scattering medium 1-1 when an image is taken and allows for higher depths to be imaged entirely.

The optical subsystem 1-4 may include of a cascaded slow light amplifier 2, an example is illustrated in FIG. 2. This cascaded slow light amplifier 2 may include at least one host crystal doped with ions or be multiple such crystals spliced or sandwiched together. This population of dopant ions then has its ion population treated spatially and spectrally to include spatially subsequent amplification 2-1 and filtration zones 2-2. The filtration zones may include the slow light filters specified in the previously submitted application System and method for localising light in light-scattering media.

The slow-light filter may be comprised of a at least one host crystal doped with ions with strong optical absorption at the original frequency of the optical source 1-2. As the ions replace other atoms in the host crystal, they will slightly distort its crystal lattice. While the individual absorption of these ions is strong and narrow in frequency, the entire ensemble of doped ions will see different crystal fields and thus slightly change at what frequency they absorb in relation to each other. The multitude of different ion classes yield a total absorption profile of the ensemble of doped ions in the host crystal that is two to three orders of magnitude wider in frequency than any frequency shift induced by the acoustic field.

In this absorption profile it is possible to construct different enduring spectral structures by optical pumping techniques. This optical pumping may be performed with or without the application of electric and/or magnetic fields to split energy levels of the ions. A side effect of this modified absorption profile is that the frequency shifted light may experience a reduction in group velocity by several orders of magnitude. As such, the light which is shifted by the acoustic field may be additionally differentiated from the original optical frequency via time gating.

The amplification zones may be created by momentarily inverting the population of the ions. In such a state, the inverted ions amplify the field via stimulated emission. Moreover, timing the inversion of the population to the light field propagation through the amplifier allows for a diminished noise contribution from amplified spontaneous emission (ASE). By further applying electrical fields, amplification depletion at the wanted wavelength may be circumvented.

As described above, light with both the original optical frequency f1 and the by the acoustic field shifted frequency f3 enters the collection waveguide 1-7. This light is guided to the optical subsystem 1-4 which is comprised of a cascaded slow light amplifier. The frequency difference between f1 and f3 may be on the scale of 10-6 Hz. To select only f3 for amplification, very sharp optical transitions must be used so that f1 is not amplified simultaneously. Such sharp gain profiles may be achieved in ion doped crystals, such as Pr: YSO. Each ion doped into the crystal individually has a very sharp optical transition while the inhomogeneities of the crystal shifts their transition center frequency. The ensemble of all ions doped into the crystal has a very high absorption which may cover a >10-9 Hz wide frequency span. Simultaneously single ions may be targeted inside this ensemble, which with optical pumping schemes may be used to create structures in the spectral domain with a resolution of the ion transition linewidth, which is on the scale of 10-4 Hz.

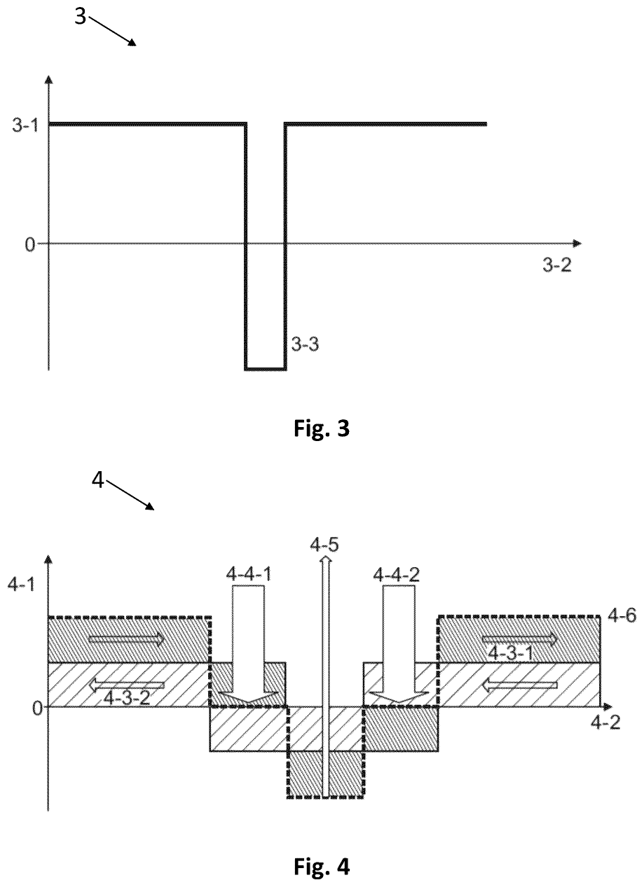

The slow light filters may, as described above and in System and method for localising light in light-scattering media, be constructed in these materials by moving ions in the ensemble to other long-lived ground states. However, the ions may also be excited to an excited state which, due to the long narrow linewidths of the transition, often also have long lifetimes, e.g. 10\hat{ }−5 to 10\hat{ }−2 s. If more than half the population can be placed in the excited state, incoming light stimulates emission and optical amplification occurs. In normal optical amplifiers this population inversion requires more than two energy levels as ions are continuously excited to the upper state. Continual excitation via optical pumping will, due to stimulated emission, generate a steady population configuration where half is in the excited state and half in the ground state. Such a population does not exhibit optical gain as there is an equal amount of absorption as amplification. However, by applying so called pi-pulses, the total population can be inverted simultaneously and immediately. As exemplified in FIG. 3, which is a plot of the absorption 3-1 versus the frequency 3-2, such a population inversion also inverts the absorption. This negative absorption is synonymous to amplification.

Furthermore, the negative absorption in turn causes a higher dispersion due to a higher gradient in absorption. Inside an amplifier stage with such an inverted population, the slow light effect is therefore even higher than in a normal slow light filter. Light which is not being targeted for amplification, i.e. the light with frequency f1, is therefore differentiable in time from the light targeted for amplification, i.e. light with frequency f3.

As stated previously in this description, an issue with optical amplifiers is the amplification of spontaneous emission (ASE). When the population is inverted, spontaneously emitted light also stimulates emission in the amplifier. If the spontaneous emission is greater than the incoming signal, then the amplified light after the amplifier stage is dominated by ASE noise. This limits the amount of amplification that is possible to apply in each stage as increased amplification garners increased spontaneous emission. By placing multiple slow light amplifier stages in a cascading fashion, the amplification can in each stage be kept below the limit at which ASE becomes an issue. Placing an optional slow light filter in between the amplifier stages act as time buffers and frequency cleaners after the amplification stage. The time buffer also allows for the population inversion of the next amplifier stage.

A final issue to resolve stems from that the amplification is proportional to the population inversion, and therefore also the total population of ions. To maximize the amplification requires many ions to be inverted, which is limited by the number of ions per amplification stage and frequency. The population may be increased by either increasing the physical length of the amplification stage or, for low dopant levels, increase the dopant concentration. A third way of increasing the number of ions contributing to the amplification is by application of an electric field. Under the influence of an external electric field, the ions experience the Stark effect and have their transition frequencies shifted. Depending on the Stark coefficients of the individual ions, this shift either increases or decreases their transition center frequency. By first inverting ions in a wide frequency region and then applying an electric field, the amplification at the center of the inverted region can be maintained by continually ramping an electric field over the amplification stage.

To continually have amplification in a frequency region, such as 3-3, two strong optical fields can be applied with frequencies above and below the targeted frequency. This is exemplified in FIG. 4, which again plots the absorption 4-1 versus the frequency 4-2 but now also splits the absorption between two ion populations 4-3 with Stark coefficients of opposite signs (Q. Li, A. Kinos, A. Thuresson, L. Rippe and S. Kroll, “Using electric fields for pulse compression and group-velocity control”, Phys. Rev. A 95, 032104 (2017)). The strong optical fields 4-4 are applied on both sides of the amplified optical field 4-5, inverting the ion population. The electric field then moves the different ion populations 4-3 in opposite directions via the Stark effect. The two populations meet in frequency space to generate amplification for the signal optical field 4-5. This scheme allows for continuous supply of fully inverted populations, i.e. maximum amplification, for each roundtrip through the amplifier. Not performing this regeneration of the inverted population would slowly decrease the amplification for each roundtrip until the steady population state, i.e. half in the excited state and half in the ground state, is reached and where pi-pulses would not change the net population distribution.

If the correct electric field is applied to a slow light filter while an optical pulse is propagating inside its transmission window, it is possible to shift the frequency of said optical pulse (Q. Li, Y. Bao, A. Thuresson, A. Nilsson, L. Rippe and S. Kroll, “Slow-light-based optical frequency shifter”, Phys. Rev. A 93, 043832 (2016)). By applying such an electric field to the final filtration zone 2-3, the light with frequency f3 may be shifted back to the original frequency f1. This resets the frequency state of the light to its input value, without removing its mode structure. Using an amplifier chain which implements both the regeneration described in FIG. 4 and this frequency shifter as the optical subsystem 1-4 in FIG. 1 allows for multiple amplifie d roundtrips through the light scattering medium 1-1 with equal amplification. These roundtrips then, as stated, allow for a build-up of the light which is transmitted in the modes 1-6-2. To facilitate the electric field across the different zones in the amplifier chain, the crystal or crystals comprising the amplifier may be connected via e.g. metal electrodes to one or multiple electric sources.

By timing the acoustic pulse repetition frequency to the amplifier roundtrip time (i.e. the time the light takes around the system loop 1-4 to 1-5 to 1-1 to 1-7 to 1-4, which due to the slow light effect in the amplifier can be several microseconds) the optical modes 1-6-2 which interacts with the acoustic field 1-3 is amplified while all other modes are suppressed. Once this has been achieved, increasing the pulse repetition frequency shifts acoustic field 1-3 deeper into the tissue for each amplifier roundtrip. If this perceived spatial shift is such that the overlap of the acoustic field in between two amplifier roundtrips is large enough, a high optical intensity is maintained at the acoustic field position even while it is continuously lowered into the tissue. This intensity is maintained as modes which mainly interact with the acoustic field 1-3 on its deeper end one roundtrip sees their frequency shifted light fraction increase on a subsequent roundtrip, making these modes stronger in the mode competition, garnering stronger amplification. Consequently, modes which interact with the shallower end of the acoustic field 1-3 are outcompeted in subsequent roundtrips and the strongest modes one roundtrip diminishes in the next.

Mismatching the acoustic pulse repetition frequency to the amplifier roundtrip thus allows for higher optical fields to reach the acoustic field deep inside the tissue, effectively increasing the imaging depth.

The present invention has been described above with reference to specific examples. However, other examples than the above described are equally possible within the scope of the disclosure. Different method steps than those described above, performing the method by hardware or software, may be provided within the scope of the invention. The different features and steps of the invention may be combined in other combinations than those described. The scope of the disclosure is only limited by the appended patent claims.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases.

1. A cascaded slow light amplifier, comprising of a unit wherein each unit comprising of at least one host crystal doped with ions and optically prepared to provide at least one amplification zone.

2. The cascaded slow light amplifier according to claim 1,
wherein said unit comprising of at least one time-filtration zone.

3. The cascaded slow light amplifier according to claim 1, wherein each zone is made of a single crystal and wherein said unit is obtained by joining at least two crystals.

4. The cascaded slow light amplifier according to claim 2, wherein a single crystal comprises said at least one amplification zone and said at least one time-filtration zone.

5. The cascaded slow light amplifier according to claim 1, comprising of a chain of a plurality of said units being linked.

6. The cascaded slow light amplifier according to claim 5, wherein said chain comprising of a plurality of said amplification zones and a plurality of said time filtration zones, wherein each of said amplification zone is subsequently followed by one of said time-filtration zone.

7. The cascaded slow light amplifier according to claim 5, wherein said chain is made from a single crystal.

8. The cascaded slow light amplifier according to claim 5, wherein a final time filter in said chain acts as a frequency shifter.

9. The cascaded slow light amplifier according to claim 8, wherein said amplification zone is a narrowband amplifier.

10. The cascaded slow light amplifier according to claim 1, wherein said amplification zone is a two-level amplifier.

11. The cascaded slow light amplifier according to claim 5, wherein said amplification zones in said chain are sequentially pumped and said amplification zones in said chain has an amplification level where a spontaneous emission (ASE) is lower than an incoming signal.

12. The cascaded slow light amplifier according to claim 6, wherein said time-filtration zone arranged between two amplification zones acts as a time buffer.

13. The cascaded slow light amplifier according to claim 1, wherein said amplification has a strong suppression outside a gain bandwidth and with enhanced slow light effect.

14. A system for regenerating an amplifier stage, comprising:
a cascaded slow light amplifier according to claim 1;
an electric source;
connectors connected to said electric source and to said amplification zones of said cascaded slow light amplifier; and
wherein said connectors are arranged to provide an electric field over one or several said amplification zones for regenerating said amplifier stage using said electric field.

15. A system for localising light in light-scattering media, said system comprising:
a light source configured for transmitting a light signal having a frequency into a light-scattering medium;
an ultrasonic device configured for generating an acoustic field in said light-scattering medium;
an optical subsystem comprising a system for regenerating an amplifier stage according to claim 14;
collection light guides configured for collecting said light signal traversed through said light scattering medium; and
wherein, when in use, said system provides an amplifier roundtrip where said light signal traverses said light-scattering medium via different optical modes, wherein some modes interact with said acoustic field whereby said modes interacting with said acoustic field has a part of its power spectrum shifted in frequency, and said light collected by said collection light guides is transmitted to said optical subsystem wherein said system for regenerating an amplifier stage amplifies frequency shifted modes and suppresses all other modes before shifting said amplified modes back to said frequency of said light signal and thereafter transmitting it back into said light-scattering medium.

16. The system of claim 15, wherein a repetition rate of said ultrasonic device is timed with a time of said amplifier roundtrip.

17. The system of claim 16, wherein said repetition rate of said ultrasonic device is incrementally changed thereby shifting said acoustic field deeper into said light-scattering medium, whereby said amplified modes follow said ultrasound field deeper into said light-scattering medium for each of said amplifier roundtrip.
